(19) United States
(12) Patent Application Publication
(10) Pub. No.: US 2013/0281846 A1
(43) Pub. Date: Oct. 24, 2013

(54) ULTRASONIC DIAGNOSTIC APPARATUS, IMAGE DISPLAY METHOD, AND IMAGE PROCESSING APPARATUS

(71) Applicants: Toshiba Medical Systems Corporation, Otawara-shi (JP); Kabushiki Kaisha Toshiba, Minato-ku (JP)

(72) Inventors: Hiroki YOSHIARA, Nasushiobara-shi (JP); Naohisa Kamiyama, Utsunomiya-shi (JP); Tetsuya Yoshida, Bergschenhoek (NL); Takeshi Sato, Nasushiobara-shi (JP)

(21) Appl. No.: 13/918,160

(22) Filed: Jun. 14, 2013

Related U.S. Application Data
(63) Continuation of application No. PCT/JP2013/057708, filed on Mar. 18, 2013.

(30) Foreign Application Priority Data
Apr. 12, 2012 (JP) 2012-091024

(51) Int. Cl. A61B 8/00; A61B 8/06; A61B 8/13; A61B 8/08

(52) U.S. Cl. CPC A61B 8/463; A61B 8/481; A61B 8/06; A61B 8/4444; A61B 8/54; A61B 8/5223; A61B 8/5207; A61B 8/13
USPC 600/431

(57) ABSTRACT

According to one embodiment, a scanning unit scans the inside of an object administered with a contrast agent with ultrasonic waves. A signal generation unit outputs a packet signal based on the reception signal output from the scanning unit. A first wall filter has a passband corresponding to a blood flow component. A second wall filter has a passband corresponding to a tissue perfusion component and blood flow component. A maximum value holding computation processing unit applies maximum value holding computation processing to a first image corresponding to an output from the first wall filter. A display unit displays the first image having undergone maximum value holding computation processing and a second image corresponding to an output from the second wall filter.

### CROSS REFERENCE TO RELATED APPLICATIONS

This application is a Continuation application of PCT Application No. PCT/JP2013/057708, filed Mar. 18, 2013 and based upon and claiming the benefit of priority from Japanese Patent Application No. 2012-091024, filed Apr. 12, 2012, the entire contents of all of which are incorporated herein by reference.

### FIELD

Embodiments described herein relate generally to an ultrasonic diagnostic apparatus, image display method, and image processing apparatus which display the microstructures of tissue perfusion and vascular flows in a contrast echo method performed by using an ultrasonic contrast agent.

### BACKGROUND

Ultrasonic diagnosis allows an operator to display in real time how the heart beats or the fetus moves, by simply bringing an ultrasonic probe into contact with the body surface. In addition, this technique is highly safe, and hence allows repeated examination. Furthermore, this system is smaller in size than other diagnostic apparatuses such as X-ray, CT, and MRI apparatuses and can be moved to the bedside to be easily and conveniently used for examination.

Ultrasonic diagnostic apparatuses used in this ultrasonic diagnosis vary in type depending on the functions which they have. Some compact apparatuses which have already been developed are small enough to be carried with one hand, and ultrasonic diagnosis is free from the influence of radiation exposure unlike diagnosis using X-rays and the like. Therefore, such ultrasonic diagnostic apparatuses can be used in obstetric treatment, treatment at home, and the like.

Recently, an intravenous ultrasonic contrast agent has been commercialized, and the contrast echo method has been performed. The purpose of this contrast echo method is to perform hemodynamic evaluation by intravenously injecting an ultrasonic contrast agent to enhance a blood flow signal in examination of the heart, liver, or the like.

Many types of contrast agents are designed such that microbubbles function as reflection sources. In this case, the base material is air bubbles which have delicate characteristics. For this reason, even at ultrasonic irradiation at a general diagnostic level, air bubbles sometimes collapse due to a corresponding mechanical effect. This eventually decreases the intensity of signals from a scan plane.

In order to observe the dynamic state of tissue perfusion in real time, therefore, it is necessary to relatively reduce the collapse of air bubbles due to scanning, for example, by imaging with ultrasonic transmission of a low sound pressure. However, imaging by such ultrasonic transmission with a low sound pressure will decrease the signal/noise ratio (to be referred to as an S/N ratio hereinafter). For this reason, various types of signal processing methods for compensating for this decrease in S/N ratio have been proposed. This makes it possible to implement real-time visualization with a high S/N ratio.

Using the above contrast agent, however, will visualize not only a blood flow but also tissue perfusion at a capillary level. Although this is useful as diagnostic information, the blood flow is buried in tissue perfusion to degrade the visibility of a blood flow structure (blood vessel structure).

In contrast to this, the first technique has been proposed as follows, which uses the characteristic that air bubbles of the above contrast agent collapse. The first technique includes (a) observing the dynamic state of air bubbles filling a scan slice under low sound pressure irradiation, (b) making air bubbles collapse within the slice (strictly, the irradiation volume) upon switching the irradiation sound pressure to high sound pressure, and (c) observing the state of air bubbles entering the slice again. This first technique is called a replenishment (reperfusion) method. There has also been proposed an image processing method which reconstructs a minute blood vessel image by performing maximum value holding computation for an image (its luminance) during reperfusion to improve the visibility of a minute blood vessel in which flowing air bubbles are very sparse. This technique can provide tissue perfusion and a blood vessel structure as diagnostic information.

The second technique using a Doppler method has been known as an imaging method for separating tissue perfusion from blood flow information. The second technique calculates the Doppler shift of a contrast agent signal to display tissue perfusion in which the flow velocity or the like is low and a blood flow signal exhibiting a high flow velocity as compared with the tissue perfusion in different hues. This technique can improve the visibility of a blood flow as compared with a general grayscale-based image.

Recently, researches and developments of a contrast agent have been conducted for visualization or medical treatment of molecules specifically expressed in tumors and the like. For example, this contrast agent have on its surfaces special factors (ligands) for specifically adsorbing targets, and are configured to adsorb specific targets according to the types of ligands. The most advanced study on such a contrast agent is a contrast agent having a ligand targeted to VEGFR2 (vascular endothelial growth factor receptor). VEGFR2 is expressed in blood vessel cells damaged by myocardial infarction or the like and can promote revascularization. It is known that this contrast agent aggregates at targets in about several to ten minutes after the intravenous injection of the contrast agent.

Note that in a time zone of several minutes immediately after the injection of a contrast agent, the contrast agent is perfused in the body, as is known from general contrast examination. On the other hand, after a lapse of 10 minutes from the injection of the contrast agent, although the contrast agent perfused in the body disappears, the contrast agent (to be written as a targeting contrast agent hereafter) adsorbed on the target is adsorbed on a tumor, thereby further providing diagnostic information from the amount of contrast agent adsorbed and the like.

Even when using the above targeting contrast agent, tissue perfusion information and blood flow information are important as diagnostic information.

However, high sound pressure transmission for reperfusion in the first technique described above destroys the targeting contrast agent (target bubbles) adsorbed on a target, and hence cannot be used in the process of adsorbing the targeting contrast agent.

Even when the second technique is used, since a minute blood flow (structure) is buried in tissue perfusion or is influenced by motion artifacts, it is difficult to improve the visibility of the microstructure of a vascular flow.

### BRIEF DESCRIPTION OF THE DRAWING

FIG. 1 is a block diagram showing the arrangement of an ultrasonic diagnostic apparatus 10 according to the first embodiment.

FIG. 2 is a block diagram for explaining the details of an image generation circuit 24 shown in FIG. 1.

FIG. 3 is a flowchart showing a processing procedure by the ultrasonic diagnostic apparatus 10 according to this embodiment.

FIG. 4 is a block diagram for explaining an example of the flow of a signal when a contrast mode is set in the ultrasonic diagnostic apparatus 10 according to this embodiment.

FIG. 5 is a block diagram for explaining an example of the flow of a signal when a blood flow mode is set in the ultrasonic diagnostic apparatus 10 according to this embodiment.

FIG. 6 is a view showing an example of the detection of a motion artifact frame.

FIG. 7 is a view showing an example of transition between display images upon switching from a contrast mode to a blood flow mode in this embodiment.

FIG. 8 is a flowchart showing a processing procedure by an ultrasonic diagnostic apparatus 10 according to the second embodiment.

FIG. 9 is a view showing an example of transition between display images upon switching from a contrast mode to a blood flow mode in this embodiment.

### DETAILED DESCRIPTION

In general, according to one embodiment, an ultrasonic diagnostic apparatus according to this embodiment comprises an ultrasonic probe, scanning unit, signal generation unit, first wall filter, second wall filter, maximum value holding computation processing unit, and display unit. The scanning unit scans the inside of an object administered with a contrast agent with ultrasonic waves via the ultrasonic probe. The signal generation unit generates a quadrature detection signal based on the reception signal output from the scanning unit and outputs a packet signal constituted by a plurality of quadrature detection signals. The first wall filter has a passband corresponding to a blood flow component included in the packet signal. The second wall filter has a passband corresponding to a tissue perfusion component and blood flow component included in the packet signal. The maximum value holding computation processing unit applies maximum value holding computation processing to a first image corresponding to an output from the first wall filter.

The display unit displays the first image having undergone maximum value holding computation processing and a second image corresponding to an output from the second wall filter.

Ultrasonic diagnostic apparatuses according to the first and second embodiments will be described below with reference to the accompanying drawings. Note that the same reference numerals denote constituent elements having almost the same functions and arrangements in the following description, and a repetitive description will be made only when required.

### First Embodiment

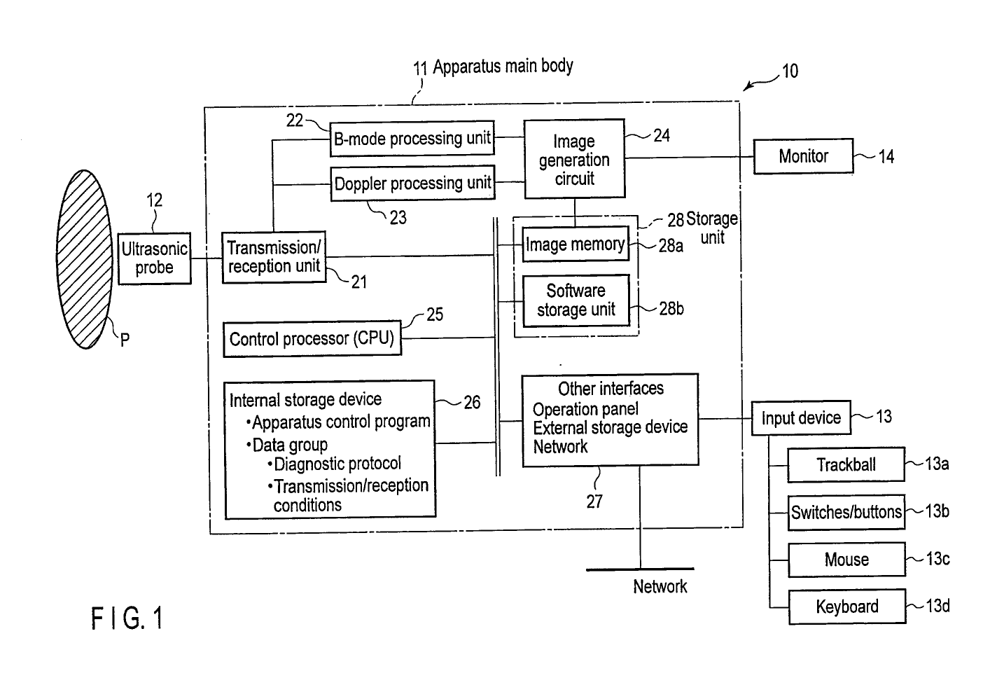

The first embodiment will be described first. FIG. 1 is a block diagram showing the arrangement of an ultrasonic diagnostic apparatus 10 according to the first embodiment. As shown in FIG. 1, the ultrasonic diagnostic apparatus 10 includes an ultrasonic diagnostic apparatus main body (to be simply referred to as an apparatus main body hereinafter) 11, an ultrasonic probe 12, an input device 13, and a monitor 14. The apparatus main body 11 includes a transmission/reception unit 21, a B-mode processing unit 22, a Doppler processing unit 23, an image generation circuit 24, a control processor (CPU) 25, an internal storage device 26, an interface unit 27, and a storage unit 28 including an image memory 28a and a software storage unit 28b. Note that the transmission/reception unit 21 and the like incorporated in the apparatus main body 11 are sometimes implemented by hardware such as integrated circuits and other times by software programs in the form of software modules. The function of each constituent element will be described below.

The ultrasonic probe 12 includes a plurality of piezoelectric transducers which generate ultrasonic waves based on driving signals from the transmission/reception unit 21 and convert reflected waves from an object P into electrical signals, a matching layer provided for the piezoelectric transducers, and a backing member which prevents ultrasonic waves from propagating backward from the piezoelectric transducers. When the ultrasonic probe 12 transmits an ultrasonic wave to the object P, the transmitted ultrasonic wave is sequentially reflected by a discontinuity surface of acoustic impedance of internal body tissue, and is received as an echo signal by the ultrasonic probe 12. The amplitude of this echo signal depends on an acoustic impedance difference on the discontinuity surface by which the echo signal is reflected. The echo produced when a transmitted ultrasonic pulse is reflected by the surface of a moving blood flow, cardiac wall, or the like is subjected to a frequency shift depending on the velocity component of the moving body in the ultrasonic transmission direction due to the Doppler effect.

The input device 13 is connected to the apparatus main body 11 and includes a trackball 13a, various types of switches and buttons 13b, a mouse 13c, and a keyboard 13d which are used to input, to the apparatus main body 11, various types of instructions, conditions, an instruction to set a region of interest (ROI), various types of image quality condition setting instructions, and the like from an operator.

The monitor 14 displays morphological information and blood flow information in the living body as images based on video signals from the image generation circuit 24.

The transmission/reception unit 21 includes a trigger generation circuit, delay circuit, and pulser circuit (none of which are shown). The pulser circuit repetitively generates rate pulses for the formation of transmission ultrasonic waves at a predetermined rate frequency fr Hz (period: 1/fr sec). The delay circuit gives each rate pulse a delay time necessary to focus an ultrasonic wave into a beam and determine transmission directivity for each channel. The trigger generation circuit applies a driving pulse to the ultrasonic probe 12 at the timing based on this rate pulse.

The transmission/reception unit 21 has a function of instantly changing a transmission frequency, transmission driving voltage, or the like in accordance with an instruction from the control processor 25. In particular, the function of changing a transmission driving voltage is implemented by linear amplifier type transmission circuit capable of instantly switching its value or a mechanism of electrically switching a plurality of power supply units.

The transmission/reception unit 21 includes an amplifier circuit, A/D converter, and adder (none of which are shown). The amplifier circuit amplifies an echo signal received via the ultrasonic probe 12 for each channel. The A/D converter gives the amplified echo signals delay times necessary to determine reception directivities. The adder then performs addition processing for the signals. With this addition, a reflection component from a direction corresponding to the reception directivity of the echo signal is enhanced to form a composite beam for ultrasonic transmission/reception in accordance with reception directivity and transmission directivity.

The B-mode processing unit 22 receives an echo signal from the transmission/reception unit 21, and performs logarithmic amplification, envelope detection processing, and the like for the signal to generate data whose signal intensity is expressed by a luminance level. This data is transmitted to the image generation circuit 24. The monitor 14 displays the data as a B-mode image whose reflected wave intensity is expressed by a luminance.

The Doppler processing unit 23 frequency-analyzes velocity information from the echo signal received from the transmission/reception unit 21 to extract a blood flow, tissue, and contrast agent echo component by the Doppler effect, and obtains blood flow information such as an average velocity, variance, and power at multiple points. The obtained blood flow information is sent to the image generation circuit 24, and is displayed in color as an average velocity image, variance image, power image, and combined image of them on the monitor 14.

The image generation circuit 24 generates an ultrasonic diagnostic image as a display image by converting the scanning line signal string for ultrasonic scanning into a scanning line signal string in a general video format typified by a TV format. The image generation circuit 24 incorporates a memory for storing image data, and allows the operator to read out a recorded image during examination after diagnosis. Note that data before it is input to the image generation circuit 24 is sometimes called “raw data”.

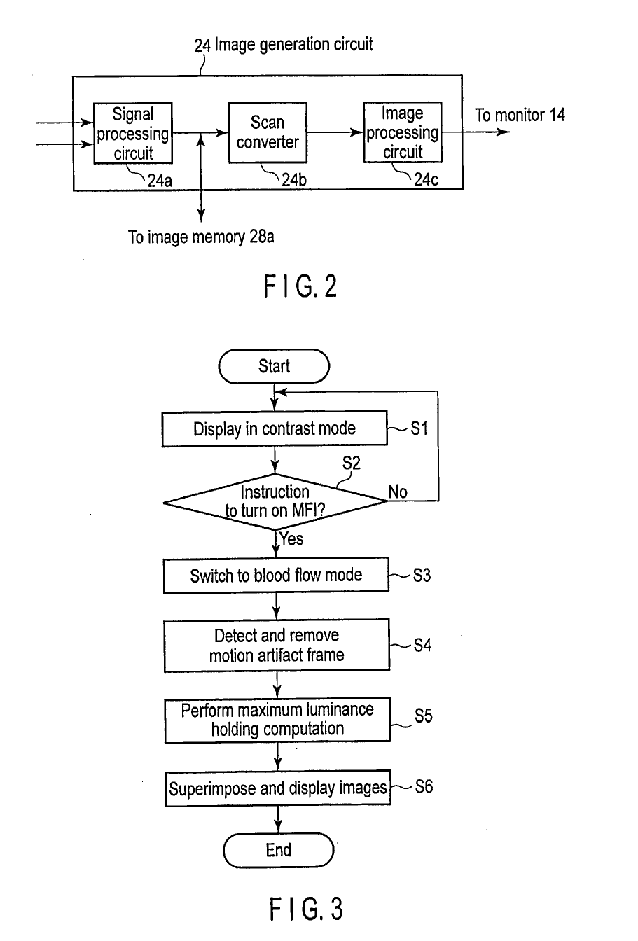

FIG. 2 shows the details of the image generation circuit 24. As shown in FIG. 2, the image generation circuit 24 includes a signal processing circuit 24a, a scan converter 24b, and an image processing circuit 24c.

First of all, the signal processing circuit 24a performs filtering to determine image quality at the scanning line level in ultrasonic scanning. An output from the signal processing circuit 24a is sent to the scan converter 24b and stored in the image memory 28a in the storage unit 28.

The scan converter 24b converts the scanning line signal string for ultrasonic scanning into a scanning line signal string in a general video format typified by a TV format. An output from the scan converter 24b is sent to the image processing unit 24c.

The image processing unit 24c executes image processing such as adjustment of a luminance and contrast and spatial filtering or combines the generated image with character information of various types of set parameters, scale marks, and the like, and outputs the resultant data as a video signal to the monitor 14. The monitor 14 then displays a tomographic image indicating the tissue form of the object.

The control processor 25 has the function of an information processing apparatus (computer) and controls the operation of the apparatus main body 11. The control processor 25 reads out a control program for implementing ultrasonic transmission/reception, image generation, image display, and the like (to be described later) from the internal storage device 26, expands the program in the software storage unit 28b in the storage unit 28, and executes computation, control, and the like associated with each type of processing.

The internal storage device 26 stores, for example, the above control program, diagnosis information (patient ID, findings by doctors, and the like), a diagnostic protocol, transmission/reception conditions, and other data groups. The internal storage device 26 is also used to archive images in the image memory 28a, as needed. It is possible to transfer data in the internal storage device 26 to an external peripheral device of the ultrasonic diagnostic apparatus 10 via the interface unit (interface circuit) 27.

The interface unit 27 is an interface associated with the input device 13, a network, and a new external storage device (not shown). The interface unit 27 can transfer, via a network, data such as ultrasonic images, analysis results, and the like obtained by the ultrasonic diagnostic apparatus 10.

Note that the image memory 28a described above is formed from a memory which stores the image data received from the signal processing circuit 24a. For example, the operator can read out this image data after diagnosis, and can reproduce the data as a still image or a moving image by using a plurality of frames. The image memory 28a also stores an output signal (called a radio frequency (RF) signal) immediately after it is output from the transmission/reception unit 21, an image luminance signal immediately after it is transmitted through the B-mode processing unit 22 and the Doppler processing unit 23, other raw data, image data acquired via a network, and the like, as needed.

The operation of the ultrasonic diagnostic apparatus 10 according to this embodiment will be described next. The ultrasonic diagnostic apparatus 10 according to this embodiment sets either the blood flow mode (first mode) or the contrast mode (second mode), which will be described later, in accordance with an instruction from the operator. The ultrasonic diagnostic apparatus 10 operates in accordance with the set mode. The control processor 25 included in the apparatus main body 11 of the ultrasonic diagnostic apparatus 10 according to the embodiment has a function of controlling the operation of the image generation circuit 24 to switch between the blood flow mode and the contrast mode.

Assume that this embodiment uses a contrast agent such as target bubbles. That is, the ultrasonic diagnostic apparatus 10 according to the embodiment scans the inside of the object P administered with a contrast agent (for example, target bubbles) with ultrasonic waves via the ultrasonic probe 12.

A processing procedure by the ultrasonic diagnostic apparatus 10 according to this embodiment will be described with reference to the flowchart of FIG. 3. Assume that the contrast mode is set in the ultrasonic diagnostic apparatus 10.

In this case, the ultrasonic diagnostic apparatus 10 displays an image corresponding to the contrast mode with low sound pressure (step S1). Note that the contrast mode is a mode for visualizing a blood flow or tissue perfusion by, for example, a grayscale-based or Doppler-based processing. Note that the concrete flow of a signal in a case in which the contrast mode is set will be described later.

In this case, the operator can issue an instruction to switch to the blood flow mode (that is, turn on MFI) via, for example, a command screen or operation panel. Without any such instruction from the operator (NO in step S2), the apparatus keeps performing the processing in step S1, that is, keeps displaying an image corresponding to the contrast mode.

With such an instruction from the operator (instruction to turn on MFI) (YES in step S2), the control processor 25 included in the apparatus main body 11 switches the contrast mode set in the ultrasonic diagnostic apparatus 10 to the blood flow mode (step S3). Note that the blood flow mode is a mode in which, for example, transmission/reception conditions (a reception band, PRF, and the like) and a wall filter are suitably set to suitably extract a contrast agent flowing at a relatively high flow velocity.

In this case, the image generation circuit 24 included in the apparatus main body 11 described above includes the first wall filter having a passband corresponding blood flow components included in signals such as average velocity, variance, and power signals and the second wall filter having a passband corresponding to tissue perfusion components and blood flow components included in the signals. Since a vascular flow is higher in flow velocity than tissue perfusion, the first wall filter has a function of extracting a signal from a contrast agent flowing at a relatively high flow velocity in a region of interest (moving relative to the region of interest). In contrast, the second wall filter has a function of extracting a signal from a contrast agent flowing at a relatively low flow velocity in the region of interest (resting relative to the region of interest) and a signal from a contrast agent flowing at a relatively high flow velocity in the region of interest.

If the contrast mode is switched to the blood flow mode as described above (that is, the blood flow mode is set in the ultrasonic diagnostic apparatus 10), the image generation circuit 24 generates a blood flow image (first image) corresponding to an output from the first wall filter and a tissue perfusion image (second image) corresponding to an output from the second wall filter. Note that a blood flow image is an image for displaying a vascular flow in the region of interest, and a tissue perfusion image is an image for displaying tissue perfusion and a vascular flow in the region of interest.

In this case, when capturing an image of a minute blood flow at a low flow velocity by, for example, Doppler-based processing, the image tends to be influenced by motion artifacts. This may degrades a blood flow image which is a maximum luminance holding image (to be described later). For this reason, the image generation circuit 24 detects a motion artifact frame in a blood flow image corresponding to an output from the first wall filter and removes the motion artifact frame (step S4). For example, a motion artifact frame is detected based on the displacements between frames in a blood flow image based on the velocity information or tissue image of each frame.

The image generation circuit 24 then applies maximum luminance holding computation processing (maximum value holding computation processing) to the blood flow image subjected to the above motion artifact frame detection and removal processing (step S5). This maximum luminance holding computation processing is the processing of generating a new image by, for example, selecting the maximum value of luminance values spatially corresponding to a plurality of frames.

Note that the apparatus may combine the processing in steps S4 and S5 described above with processing such as motion correction processing of correcting the positional shifts between frames. Combining such types of processing makes it possible to generate an image with high visibility of a blood flow structure (maximum luminance holding image).

Upon executing the processing in step S5, the image generation circuit 24 generates a display image with the maximum luminance holding image (the blood flow image having undergone the maximum luminance holding computation processing) being superimposed on the real-time tissue perfusion image corresponding to an output from the second wall filter. For example, the monitor 14 displays the display image generated in this case (step S6). When generating a display image, it is also possible to adjust the dynamic range, gain, map, and like of a blood flow image having undergone maximum luminance holding computation processing suitably for blood flow visibility. In this embodiment, this makes it possible to simultaneously display (provide) a blood vessel microstructure and tissue perfusion as diagnostic images.

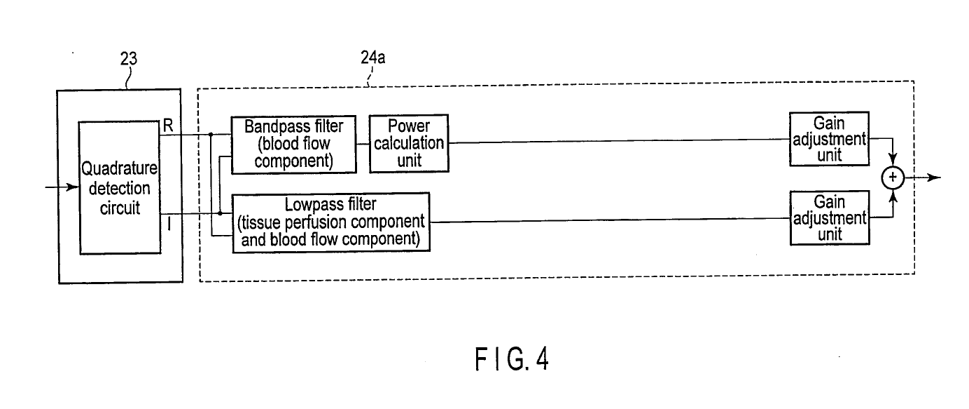

An example of the flow of a signal in a case in which the contrast mode is set in the ultrasonic diagnostic apparatus 10 according to this embodiment will be described next with reference to FIG. 4. The flow of a signal in the Doppler processing unit 23 and the image generation circuit 24 will be mainly described below.

A signal input to the Doppler processing unit 23 (that is, a signal transferred from the transmission/reception unit 21 to the Doppler processing unit 23) will be described first. A signal input to the Doppler processing unit 23 includes a signal with a suppressed fundamental wave component and an enhanced second harmonic (second harmonic wave) component as a nonlinear signal. Note that the transmission/reception unit 21 obtains this signal by transmitting a signal having a second waveform (waveform with an inverted amplitude) 180° out of phase from the first transmission waveform and adding the resultant echo signal (reflected wave data).

When such a signal is input to the Doppler processing unit 23, the quadrature detection circuit included in the Doppler processing unit 23 shown in FIG. 4 detects a complex signal (quadrature detection signal) constituted by a real part (R) and an imaginary part (I) by performing quadrature detection for the signal. Note that quadrature detection is performed by mixing the signal input to the Doppler processing unit 23 with an in-phase signal or a signal 90° out of phase. A set of quadrature detection signals extracted by the quadrature detection circuit in this manner are sent as a packet signal to the image generation circuit 24. Note that a packet signal is a set of a plurality of IQ signals.

As described above, when the contrast mode is set, the first wall filter (bandpass filter) in the image generation circuit 24 (signal processing circuit 24a) extracts a signal based on a contrast agent flowing in a region of interest at a relatively high flow velocity from the packet signal constituted by the above quadrature detection signals, and the second wall filter (lowpass filter) in the image generation circuit 24 (signal processing circuit 24a) extracts a signal based on a contrast agent flowing in the region of interest at a relatively low flow velocity and a signal based on a contrast agent flowing in the region of interest at a relatively high flow velocity. In this case, the bandpass filter is set so as not to include any clutter component (component with no frequency shift) in the passband.

Note that the signal extracted by the first wall filter (the signal based on the contrast agent flowing in the region of interest at a relatively high flow velocity) is, for example, a signal based on a blood flow component included in a packet signal. The signals extracted by the second wall filter (the signal based on the contrast agent flowing in the region of interest at a relatively low flow velocity and the signal based on the contrast agent flowing in the region of interest at a relatively high flow velocity) are, for example, signals based on a tissue perfusion component and blood flow component included in the packet signal. In the following description, for the sake of convenience, the signal extracted by the first wall filter (obtained by filtering a packet signal with the first wall filter) will be referred to as a blood flow signal, and the signal extracted by the second wall filter (obtained by filtering a packet signal with the second wall filter) will be referred to as a tissue perfusion signal. That is, a blood flow signal is obtained by filtering a packet signal with the first wall filter, and a tissue perfusion signal is obtained by filtering a packet signal with the second wall filter.

In the signal processing circuit 24a, a power calculation unit calculates the power of a blood flow signal. Note that the power of the blood flow signal is calculated by R²+I² where R is the real part of the signal and I is the imaginary part of the signal.

The gain adjustment unit of the signal processing circuit 24a then performs gain adjustment and the like for the blood flow image corresponding to the blood flow signal whose power is calculated and the tissue perfusion image corresponding to the tissue perfusion signal to generate a display image based on the gain-adjusted blood flow image and tissue perfusion image.

Note that when performing gain adjustment, the apparatus performs processing such as weighting the blood flow image and tissue perfusion image for the generation of a display image. That is, a display image depends on a gain adjustment processing result. In the contrast mode, when performing gain adjustment processing, for example, a blood flow image weight (w1) is set to be equal to a tissue perfusion image weight (w2) (w1≈w2). In the contrast mode, this generates a display image with the same ratio between a blood flow image and a tissue perfusion image.

As described above, when the contrast mode is set, the apparatus generates a display image based on a blood flow image corresponding to a blood flow signal and a tissue perfusion image corresponding to a tissue perfusion signal to visualize both the blood flow and the tissue perfusion. In some cases, the blood flow structure is buried in the tissue perfusion, and hence the visibility of the blood flow structure is low.

In the case shown in FIG. 4, when the contrast mode is set, the apparatus processes both a blood flow signal and a tissue perfusion signal. However, when the contrast mode is set, the apparatus may process, for example, only a tissue perfusion signal (i.e., display only the tissue perfusion image). Alternatively, the apparatus may be configured to perform the same processing for a signal before it is separated into a blood flow signal and a tissue perfusion signal by the corresponding filters.

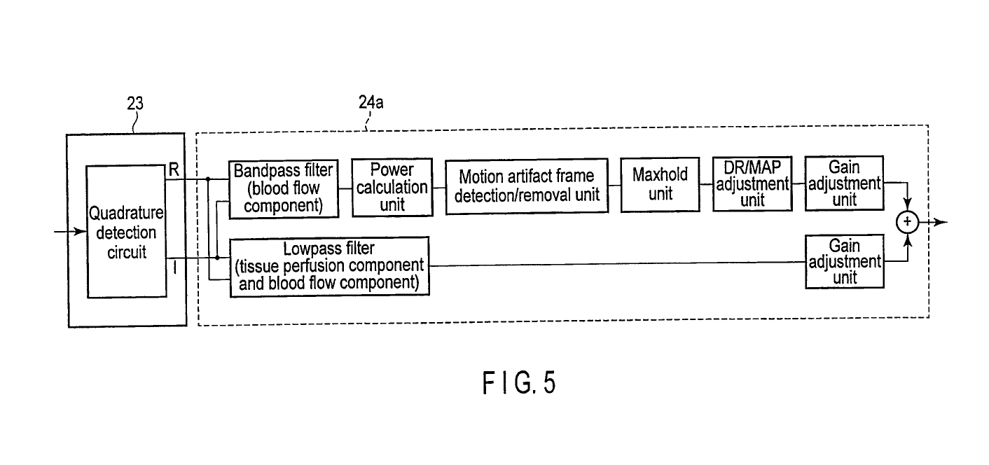

An example of the flow of a signal in a case in which the blood flow mode is set in the ultrasonic diagnostic apparatus 10 according to this embodiment will be described next with reference to FIG. 5. As in the case shown in FIG. 4 described above, the following will mainly describe the flow of a signal in the Doppler processing unit 23 and the image generation circuit 24. Note that the flow of a signal in the Doppler processing unit 23 is the same as that in the case in which the contrast mode is set, and hence a detailed description of this will be omitted.

When the blood flow mode is set, the first wall filter (bandpass filter) in the image generation circuit 24 (signal processing circuit 24a) extracts a signal (blood flow signal) based on a contrast agent flowing in a region of interest at a relatively high flow velocity from the above packet signal, and the second wall filter (lowpass filter) in the image generation circuit 24 (signal processing circuit 24a) extracts a signal based on a contrast agent flowing in the region of interest at a relatively low flow velocity and a signal based on a contrast agent flowing in the region of interest at a relatively high flow velocity (tissue perfusion signal).

The following will describe the processing performed for a blood flow signal (to be referred to as processing on the blood flow signal side hereinafter) and the processing performed for a tissue perfusion signal (to be referred to as processing on the tissue perfusion signal side hereinafter) in a case in which the blood flow mode is set.

Processing on the blood flow side will be described first. In this case, in the signal processing circuit 24a, the power calculation unit calculates the power of a blood flow signal. The power calculation unit performs this power calculation processing for the blood flow signal in the same manner as that described in the case in which the contrast mode is set, and hence a detailed description of this will be omitted.

A motion artifact frame detection/removal unit in the signal processing circuit 24a then detects a motion artifact frame in a blood flow image corresponding to the blood flow signal whose power has been calculated, and removes the detected motion artifact frame.

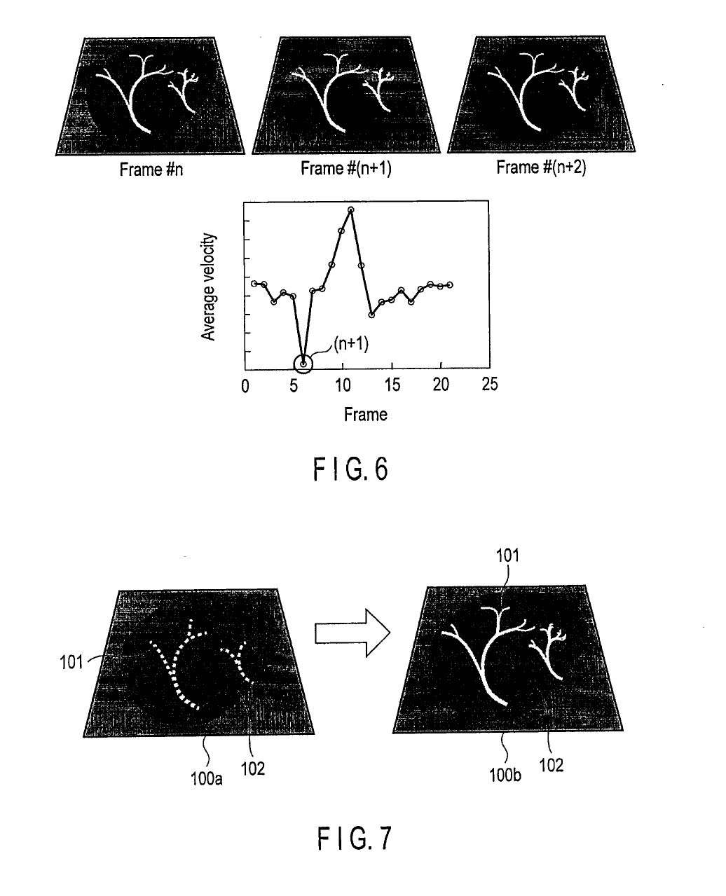

FIG. 6 shows an example of the detection of a motion artifact frame. In the case shown in FIG. 6, the apparatus monitors velocity information in the overall image or a region of interest in each of consecutive frames (blood flow images), and detects, as a motion artifact frame, a frame which exhibits a change in velocity information exceeding a threshold relative to an adjacent frame. Note that, as described above, the apparatus may also detect a motion artifact by using the displacements between frames based on, for example, a tissue image.

The maxhold unit (maximum value holding computation processing unit) of the signal processing circuit 24a applies maximum luminance holding computation processing (maxhold processing) to a blood flow image from which a motion artifact frame is removed.

The image generation circuit 24 (signal processing circuit 24a) then adjusts, for example, a dynamic range (DR) and a map (MAP) by using DR and MAP adjustment units, and performs processing such as gain adjustment described above by using a gain adjustment unit. Note that processing such as dynamic range (DR) adjustment and map (MAP) adjustment may be performed for a maximum luminance holding image (a blood flow image having undergone maximum luminance holding computation processing).

Processing on the tissue perfusion signal side will be described next. This processing on the tissue perfusion signal side is executed in the same manner as in a case in which the contrast mode is set. More specifically, gain adjustment or the like is performed for a tissue perfusion image corresponding to a tissue perfusion signal.

Upon executing the processing on the blood flow signal side and the processing on the tissue perfusion signal side, the apparatus generates a display image based on a maximum luminance holding image (a blood flow image having undergone maximum luminance holding computation processing) and a tissue perfusion image corresponding to the tissue perfusion signal.

Note that in gain adjustment processing in the blood flow mode, for example, the maximum luminance holding image weight (w1) is set to be larger than the tissue perfusion image weight (w2) (w1>w2). This generates a display image with a large ratio of a maximum luminance holding image (i.e., a blood flow image) in the blood flow mode.

As described above, when the blood flow mode is set, since maximum luminance holding computation processing has been applied to only a blood flow image by the processing on the blood flow signal side, it is possible to avoid a degradation in the visibility of a blood flow structure due to the burying of the blood flow structure in tissue perfusion and simultaneously provide the blood flow microstructure and tissue perfusion as a diagnostic image.

FIG. 7 shows an example of the transition between display images upon switching from the contrast mode to the blood flow mode in this embodiment.

In the case shown in FIG. 7, a display image 100a indicates a display image when the contrast mode is set. In contrast, a display image 100b indicates a display image when the blood flow mode is set.

As described above, when the blood flow mode is set, since maximum luminance holding computation processing is applied to only a blood flow image, it is possible to clearly observe a blood vessel structure 101 in the display image 100b, as shown in FIG. 7, as compared with the display image 100a. Note that in the display images 100a and 100b shown in FIG. 7, tissue perfusion 102 is displayed around the blood vessel structure 101.

As described above, this embodiment can display an image with improved visibility of the microstructure of a vascular flow by being configured to apply maximum value holding computation processing to a blood flow image (first image) corresponding to an output (i.e., a blood flow signal) from the first wall filter and, display the blood flow image having undergone the maximum value holding computation processing and a tissue perfusion image (second image) corresponding to an output (i.e., a tissue perfusion signal) from the second wall filter.

That is, in this embodiment, since maximum luminance holding computation processing is applied to only a blood flow image, it is possible to avoid a degradation in the visibility of a blood flow structure due to the burying of the blood flow structure in tissue perfusion.

In addition, this embodiment can generate and display an image corresponding to the mode desired by the operator by being configured to control the image generation circuit 24 to switch between the blood flow mode (first mode) of displaying at least maximum value holding image (a blood flow image having undergone maximum value holding computation processing) and the contrast mode (second mode) of displaying a tissue perfusion image in accordance with an instruction from the operator.

This embodiment also allows simultaneous observation of a blood flow structure and tissue perfusion without burying the blood flow structure in the tissue perfusion by being configured to display a display image with a maximum luminance holding image being superimposed on a tissue perfusion image in the blood flow mode.

Furthermore, this embodiment can display an image (maximum luminance holding image) improving the visibility of a blood flow structure by being configured to detect a motion artifact frame in a blood flow image, remove the detected motion artifact frame, and apply maximum luminance holding computation processing to the resultant image.

Note that this embodiment has exemplified the case in which maximum luminance holding computation processing is performed. However, the embodiment may be configured to perform the processing (e.g., temporal afterimage processing) of generating a new image by performing weighted addition of signals at position spatially corresponding to a plurality of frames instead of the maximum luminance holding computation processing. According to this temporal afterimage processing, the range (the number of frames) in which maximum value holding computation processing is performed for a plurality of frames in a blood flow image (first image) corresponding to an output from the first wall filter described above changes with time. In other words, in temporal afterimage processing, maximum value holding computation processing is applied to only latest N frames (N is a predetermined arbitrary integer) of a plurality of frames in a blood flow image corresponding to an output from the first wall filter. More specifically, assuming that N=10, if, for example, the first to 100th frames of images are captured, maximum value holding computation processing is applied to the latest 10 images (the 91st to 100th frames of images) to generate an image. If the 101st frame of an image is then captured, the image obtained by applying maximum value holding computation to the 91st to 100th frames of images is discarded, and maximum value holding computation is applied to the new latest 10 images (the 92nd to 101st frames of images) to generate an image. In temporal afterimage processing, such processing is repeated every time an image is captured.

In addition, this embodiment may be configured to singly display a blood flow or tissue perfusion as needed, for example, during examination by the ultrasonic diagnostic apparatus 10 according to the embodiment or after freezing of an image.

For the sake of convenience, this embodiment has exemplified the case in which one of the blood flow mode and the contrast mode is set. However, the embodiment may be configured to use other modes together with the above modes.

### Second Embodiment

The second embodiment will be described next. The arrangement of the blocks of an ultrasonic diagnostic apparatus according to this embodiment is the same as that in the first embodiment, and hence will be described with reference to FIGS. 1 and 2, as needed.

An ultrasonic diagnostic apparatus 10 according to this embodiment differs from the apparatus according to the first embodiment in that when the blood flow mode is set, only the maximum luminance holding image described above is displayed.

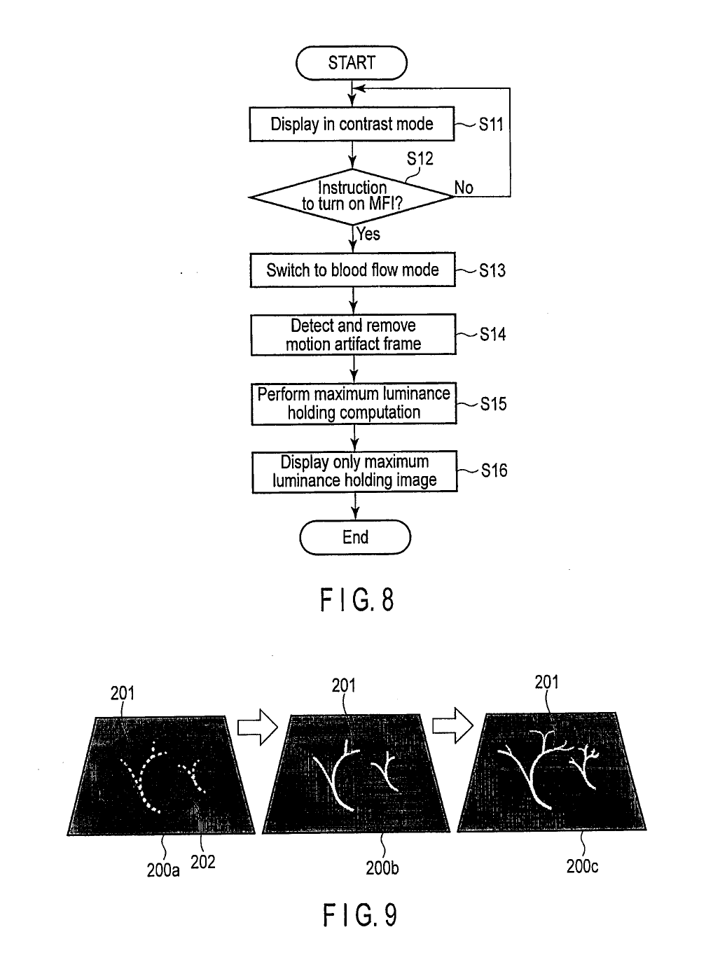

A processing procedure by the ultrasonic diagnostic apparatus 10 according to this embodiment will be described below with reference to the flowchart of FIG. 8. Assume that the contrast mode is set in the ultrasonic diagnostic apparatus 10.

In this case, the ultrasonic diagnostic apparatus 10 executes the processing in steps S11 to S15 corresponding to the processing in steps S1 to S5 in FIG. 3 described above.

An image generation circuit 24 then generates a display image based on a blood flow image (maximum luminance holding image) having undergone maximum luminance holding computation processing in step S15. For example, a monitor 14 displays the display image generated in this case (step S16). That is, in step S16, the apparatus displays the image obtained by removing a tissue perfusion image from the image (display image) displayed when the blood flow mode is set in the first embodiment described above (that is, only the blood flow represented by a maximum luminance holding image).

FIG. 9 shows an example of the transition between display images when the contrast mode is switched to the blood flow mode in this embodiment.

In the case shown in FIG. 9, a display image 200a indicates a display image when the contrast mode is set. A display image 200b indicates a display image immediately after the contrast mode is switched to the blood flow mode. A display image 200c indicates a display image obtained after maximum luminance holding computation processing upon switching to the blood flow mode.

That is, in this embodiment, when the contrast mode is set, the display image 200a is displayed. Thereafter, the display image 200a shifts to the display image 200b immediately after switching to the blood flow mode. In this case, tissue perfusion 202 displayed in the display image 200a is removed from the display image 200b. In the blood flow mode, after maximum luminance holding computation processing, the display image shifts to the display image 200c. The display image 200c displays, for example, sparse peripheral vessels and microscopic blood vessels more clearly than the display image 200b.

As described above, in this embodiment, when the blood flow mode is set, the apparatus generates a display image based on only a maximum luminance holding image, and hence can further improve the visibility of a blood flow structure.

In addition, in this embodiment, the apparatus can display only a blood flow structure by removing a tissue perfusion image from a display image instead of destroying bubbles (air bubbles) in a slice by, for example, the replenishment (reperfusion) method, and hence is useful when the apparatus uses target bubbles or the operator does not want to destroy a contrast agent (bubbles) more than necessary because of a small perfusion amount of contrast agent.

According to the above description, in this embodiment, when switching to the blood flow mode, the apparatus generates a tissue perfusion image based on a tissue perfusion signal as in the first embodiment. As described above, however, since a display image based on only a maximum luminance holding image is generated in this embodiment, the processing of generating this tissue perfusion image may be omitted. On the other hand, if the apparatus generates a tissue perfusion image in the same manner as in the first embodiment, the tissue perfusion image may be stored in the image memory 28a or the like so as to allow the operator to read it out after diagnosis.

According to these embodiments, it is possible to provide an ultrasonic diagnostic apparatus and program which can display an image which improves the visibility of a microstructure of vascular flow.

In addition, the processing described in the first and second embodiments may be executed by an image processing apparatus (e.g., a workstation) outside the ultrasonic diagnostic apparatus. In this case, the image processing apparatus externally reads a quadrature detection signal (a quadrature detection signal generated based on a reception signal obtained by scanning the inside of an object administered with a contrast agent with ultrasonic waves via the ultrasonic probe) and executes the above processing based on the quadrature detection signal.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
an ultrasonic probe;
a scanning unit configured to scan inside of an object administered with a contrast agent with an ultrasonic wave via the ultrasonic probe;
a signal generation unit configured to generate a quadrature detection signal based on a reception signal output from the scanning unit and output a packet signal constituted by the plurality of quadrature detection signals;
a first wall filter having a passband corresponding to a blood flow component included in the packet signal;
a second wall filter having a passband corresponding to a tissue perfusion component and blood flow component included in the packet signal;
a maximum value holding computation processing unit configured to apply maximum value holding computation processing to a first image corresponding to an output from the first wall filter; and
a display unit configured to display the first image having undergone the maximum value holding computation processing and a second image corresponding to an output from the second wall filter.

2. The ultrasonic diagnostic apparatus of claim 1, further comprising a control unit configured to switch between a first mode of causing the display unit to display at least the first image having undergone the maximum value holding computation processing and a second mode of causing the display unit to display the second image in accordance with an instruction from an operator.

3. The ultrasonic diagnostic apparatus of claim 2, wherein the display unit displays an image with the first image having undergone the maximum value holding computation processing being superimposed on the second image.

4. The ultrasonic diagnostic apparatus of claim 1, further comprising:
a detection unit configured to detect a motion artifact frame in the first image corresponding to an output from the first wall filter,
wherein the maximum value holding computation processing unit applies maximum value holding computation processing to the first image from which the detected motion artifact is removed.

5. The ultrasonic diagnostic apparatus of claim 4, wherein the detection unit detects the motion artifact frame based on a change between frames in the first image corresponding to an output from the first wall filter.

6. The ultrasonic diagnostic apparatus of claim 1, wherein the maximum value holding computation processing unit corrects a motion between frames in the first image corresponding to an output from the first wall filter during the maximum value holding computation processing.

7. The ultrasonic diagnostic apparatus of claim 1, wherein the maximum value holding computation processing unit applies the maximum value holding computation processing to a predetermined number of latest frames of a plurality of frames of the first image corresponding to an output from the first wall filter.

8. The ultrasonic diagnostic apparatus of claim 1,
wherein the first wall filter includes a bandpass filter, and
the second wall filter includes a lowpass filter.

9. An image display method executed by an ultrasonic diagnostic apparatus which scans inside of an object administered with a contrast agent with an ultrasonic wave via an ultrasonic probe, the method comprising:
generating a quadrature detection signal based on a reception signal obtained by the scanning and outputting a packet signal constituted by the plurality of quadrature detection signals;
applying maximum value holding computation processing to a first image corresponding to an output from a first wall filter having a passband corresponding to a blood flow component included in the packet signal; and
displaying the first image having undergone the maximum value holding computation processing and a second image corresponding to an output from a second wall filter having a passband corresponding to a tissue perfusion component and blood flow component included in the packet signal.

10. An image processing apparatus comprising:
a reading unit configured to read a quadrature detection signal generated based on a reception signal obtained by scanning inside of an object administered with a contrast agent with an ultrasonic wave via an ultrasonic probe;
a signal generation unit configured to output a packet signal constituted by the plurality of read quadrature detection signals;
a first wall filter having a passband corresponding to a blood flow component included in the packet signal;
a second wall filter having a passband corresponding to a tissue perfusion component and blood flow component included in the packet signal;
a maximum value holding computation processing unit configured to apply maximum value holding computation processing to a first image corresponding to an output from the first wall filter; and
a display unit configured to display the first image having undergone the maximum value holding computation processing and a second image corresponding to an output from the second wall filter.
